(19) United States
(12) Patent Application Publication
(10) Pub. No.: US 2016/0158157 A1
(43) Pub. Date: Jun. 9, 2016

(54) ANTI-TUBERCULOSIS STABLE PHARMACEUTICAL COMPOSITION IN A FORM OF A COATED TABLET COMPRISING GRANULES OF ISONIAZID AND GRANULES OF RIFAPENTINE AND ITS PROCESS OF PREPARATION

(71) Applicant: SANOFI, Paris (FR)

(72) Inventors: Prajapati DILIP, Gujarat (IN); Prasad KUM, Hyderabad (IN); Khullar PRAVEEN, Dona Paula (IN); Kumar RAMESH, Sheohar (IN); Kumar SHAKTI, Paris (FR)

(21) Appl. No.: 14/906,876

(22) PCT Filed: Jul. 22, 2014
(86) PCT No.: PCT/EP2014/065761

§ 371 Date: Jan. 21, 2016

(30) Foreign Application Priority Data
Jul. 26, 2013 (IN) 3341/CHE/2013

(51) Int. Cl. A61K 9/24; A61K 31/496; A61K 31/4409

(52) U.S. Cl. CPC A61K 9/209; A61K 31/4409; A61K 31/496

(57) ABSTRACT

The present invention relates to an oral pharmaceutical fixed dose composition for use in the treatment of tuberculosis, said oral pharmaceutical composition comprising: a) granules comprising isoniazid and at least one intragranular excipient, b) granules comprising rifapentine and at least one intragranular excipient, and c) at least one extragranular excipient, and to its process of preparation.

### TECHNICAL FIELD OF THE INVENTION

The present invention relates to a chemically stable anti-tuberculosis pharmaceutical fixed dose composition in a form of a coated tablet comprising two active principles, namely rifapentine and isoniazid, in separated granules. The invention also provides a process of preparation of such anti-tuberculosis pharmaceutical composition.

### BACKGROUND OF THE INVENTION

The infectious disease, tuberculosis (TB), is the leading cause of death worldwide from a single human pathogen, claiming more adult lives than diseases such as acquired immunodeficiency syndrome (AIDS), malaria, diarrhea, leprosy and all other tropical diseases combined (Zumla A, Grange J. B M J (1998) 316, 1962-1964). About one third of the world's population is currently infected with Mycobacterium tuberculosis (Mtb), the disease causing agent; 10% of those infected will develop clinical diseases. Although the rate at which people are developing TB has declined, the number of cases continues to increase slowly, according to WHO figures. Hardest hit areas are in the developing world, where poverty, other diseases, and inadequate health care are factors. Killing about 1.6 million people annually, TB is the second leading infectious cause of death worldwide, after HIV/AIDS.

Currently, for effective treatment of TB, a combination of a least the following drugs, isoniazid, rifampin, and pyrazinamide are given to a patient in an initial phase of treatment for 8 weeks, during which the drugs are used in combination to kill the rapidly multiplying population of Mtb as well as to prevent the emergence of drug resistance. This initial phase of treatment is followed by a continuation phase for 24 weeks during which a combination of a least the following drugs isoniazid and rifapentine are given to patients. Such a long combination therapy is not always successful, especially in patients developing drug resistant strains. Also, compliance with the relatively long course of treatment is generally poor. Such non-compliance may lead to treatment failure resulting in development of drug resistance.

In order to control the emergence of drug resistant tuberculosis, the WHO recommends the use of fixed dose combinations (FDC) in the form of tablets which comprise, in the same formulation, two different active principles, namely isoniazid and rifapentine in fixed proportions. FDCs in the form of tablets were previously disclosed.

WO 2007/43542 in the name of SUKA PHARMACEUTICAL CO., LTD discloses a pharmaceutical composition and a kit for tuberculosis treatment. The pharmaceutical composition comprises oxazole compounds, rifapentine and isoniazid, which can be in the form of a tablet.

CN 1717912 in the name of GUANXIN CEN discloses a pharmaceutical composition comprising rifapentine and isoniazid, which can be in the form of a tablet.

CN 185728 in the name of SHUAIHUA MEDICINE SCI TECH CO discloses a sustained release formulation (implant) comprising rifapentine and isoniazid, which can be in the form of a tablet.

However, it is well known by a person skilled in the art that the use of such FDCs may reduce the bioavailability of rifapentine due to an undesirable chemical reaction with isoniazid, especially in the catalytic conditions of the acidic gastric environment (Prasad B. et al. J. Pharm. Biomed. Anal. 2006; 41:1438-1441.).

As such, there remains a need for a chemically stable anti-tuberculosis oral pharmaceutical composition comprising both rifapentine and isoniazid that can prevent the reduction of the bioavailability of the rifapentine and the undesirable chemical reaction with isoniazid.

Applicant has discovered that it was possible to provide such an oral pharmaceutical composition with a satisfactory bioavailability of both active principles by separately granulating the two active principles, and by introducing them in a pharmaceutical composition.

### OBJECTS OF THE PRESENT INVENTION

A first object of the present invention is an oral chemically stable pharmaceutical fixed dose composition for use in the treatment of tuberculosis, said oral pharmaceutical composition comprising:
a) granules comprising isoniazid and at least one intragranular excipient,
b) granules comprising rifapentine and at least one intragranular excipient, and
c) at least one extragranular excipient.

Another object of the present invention is a process for the preparation of an oral pharmaceutical composition according to the present invention, said process comprising distinct steps of granulating isoniazid and granulating rifapentine.

### INVENTION

The pharmaceutical composition according to the invention is chemically stable and suitable for the treatment of tuberculosis by oral administration.

By “chemically stable” it is meant that the total amounts of impurities formed from rifapentine is less than 8% w/w with respect to the weight of rifapentine initially present in the tablet and the total amounts of impurities formed from isoniazid is less than 2% w/w with respect to the weight of isoniazid initially present in the tablet, after storage for less than 6 months between 60% RH and 75% RH, at a temperature maintained thermostatically that encompasses the usual and customary working environment from 25° C. to 30° C.

Without being linked by any theory, it is believed that the tablets according to the present invention allow a good availability of both active substances because, due to the particular configuration of the oral pharmaceutical composition, reactions between rifapentine and isoniazid under gastric conditions are limited.

The oral pharmaceutical composition is a fixed dose composition. By “fixed-dose composition” it is meant a combination of two drugs or active ingredients presented in a single dosage unit, i.e. a tablet.

The oral pharmaceutical composition comprises two active principles, namely rifapentine and isoniazid, and pharmaceutically acceptable excipients.

More precisely, the oral pharmaceutical composition comprises granules comprising isoniazid and at least one intragranular excipient (isoniazid granules), granules comprising rifapentine and at least one intragranular excipient (rifapentine granules), and at least one extragranular excipient.

The oral pharmaceutical composition is in the form of a coated tablet. The film coating is a conventional film coating which does not confer a control release of the active principles, but which facilitates the swallowing and enhances the appearance.

The coated tablet can be a coated monolayer or a coated bilayer tablet.

According to an embodiment where the oral pharmaceutical composition is a coated bilayer tablet, one layer of the oral pharmaceutical composition comprises the isoniazid granules and at least one part of the extragranular excipients. The other layer of the oral pharmaceutical composition comprises the rifapentine granules and at least the remaining extragranular excipients.

The extragranular excipients comprise a stabilizer. The stabilizer is selected from the group comprising sodium ascorbate, sodium metabisulphite, di-sodium EDTA, butyl hydroxylated toluene, citric acid, tocopherol, butyl hydroxy anisole, ascorbic acid, tartaric acid and mixtures thereof. Preferably the extragranular is selected from sodium ascorbate, sodium metabisulphite, di-sodium EDTA and mixtures thereof.

The extragranular excipients can also comprise a compound selected from the group comprising a diluent, a disintegrant, a lubricant, a solubilizer, and mixtures thereof.

As diluent, it can be mentioned microcrystalline cellulose, pregelatinized starch, dicalcium phosphate, mannitol, and mixtures thereof, preferably microcrystalline cellulose.

As disintegrant, it can be mentioned crospovidone (cross-linked polyvinylpyrrolidone), croscarmellose, sodium starch glycollate, maize starch, low substituted hydroxypropylcellulose, alginic acid, and mixtures thereof, preferably sodium starch glycollate.

As lubricant, it can be mentioned pulverulent lubricant, for example magnesium stearate, sodium sterylfumarate, calcium stearate, stearic acid, zinc stearate, glyceryl behenate, and mixtures thereof, preferably calcium stearate.

As solubilizer, it can be mentioned sodium lauryl sulphate, Tween 80, PEG 4000, and mixtures thereof, preferably sodium lauryl sulphate.

According to a specific embodiment, the intragranular excipients present in the isoniazid granules are different from those present in the rifapentine granules.

The intragranular excipient is selected from the group comprising a diluent, a disintegrant, a solubilizer, a stabilizer, a granulation binder, and mixtures thereof.

The diluent, solubilizer, stabilizer and the disintegrant are as mentioned above. They can be identical to the diluent, solubilizer, stabilizer and disintegrant used as extragranular excipients, or they can be different.

The granulation binder can be selected from povidone, such as povidone K30 and povidone K90, hydroxypropyl cellulose, polyvinyl alcohol, maize starch, pre-gelatinized starch, and mixtures thereof, preferably povidone or pre-gelatinized starch.

The film coating may comprise hydroxypropyl methylcellulose, sodium ascorbate, di-sodium EDTA, polyvinyl acetate, lactose monohydrate, polyethylene glycol, glycerin triacetate, and pigments, preferably polyvinyl acetate, hydroxypropyl methylcellulose, di-sodium EDTA and mixture thereof.

The oral pharmaceutical composition according to the present invention may be packed in any suitable packaging, for example in a double aluminium blister packaging thanks to packing machine.

According to an embodiment, the oral pharmaceutical composition comprises from 100 mg to 400 mg of rifapentine and from 50 mg to 400 mg of isoniazid.

The treatment of the tuberculosis is a long time treatment during which the dosage regimen varies. For example, a common prescribed dosing is 600 mg twice weekly for two months, with an interval of no less than 3 consecutive days (72 hours) between doses, in combination with other anti-tuberculosis drugs up to 2 months for the initial phase of TB treatment. Said 2 months phase with 600 mg once weekly is followed by a 4 months phase by direct observation therapy with isoniazid or another appropriate antituberculous agent. A common prescribed dosing for isoniazid is 5 mg/kg up to 300 mg daily in a single dose and 15mg/kg up to 900mg/day, two to three times/week.

Due to said type of treatment, it is very useful that different tablets are available which differ from one to the other one by the ratio rifapentine/isoniazid.

According to an embodiment, the ratio of rifapentine to isoniazid is comprised from 5:1 to 1:0.5, preferably the ratio of rifapentine to isoniazid is 1:1.

More specifically, tablets according to the invention can contain 300 mg of rifapentine and 300 mg of isoniazid, 300 mg of rifapentine and 75 mg of isoniazid or 225 mg of rifapentine and 75 mg of isoniazid.

According to a preferred embodiment where the stabilizer is sodium ascorbate, the ratio of sodium ascorbate to rifapentine is comprised from 1:100 to 1:0.1, preferably from 1:70 to 1:50, more preferably is from 1:65 to 1:55, and even more preferably is 1:60.

The percentages are expressed in weight with respect to the total weight of the tablet.

According to an embodiment, the oral pharmaceutical composition comprises:
from 10% to 70%, preferably from 20% to 50%, and even more preferably from 30% to 43% of rifapentine, and
from 5% to 70% preferably from 10% to 45%, and even more preferably from 11% to 36% of isoniazid.

According to an embodiment, the oral pharmaceutical composition comprises from 0.1% to 50%, preferably from 5% to 45%, and more preferably from 13% to 42% of diluent.

According to an embodiment, the oral pharmaceutical composition comprises from 0.1% to 10%, preferably from 1% to 7%, and more preferably from 2% to 4% of disintegrant.

According to an embodiment, the oral pharmaceutical composition comprises from 0.1% to 10%, preferably from 2% to 7.5%, and more preferably from 3% to 7% of binder.

According to an embodiment, the oral pharmaceutical composition comprises from 0.1% to 1%, preferably from 0.2% to 0.9%, and more preferably from 0.25% to 0.8% of lubricant.

According to an embodiment, the oral pharmaceutical composition comprises from 0.1% to 1%, preferably from 0.3% to 0.80%, and more preferably from 0.5% to 0.7% of solubilizer.

According to an embodiment, the oral pharmaceutical composition comprises from 0.1% to 2%, preferably from 0.25% to 1.5%, and more preferably from 0.5% to 1% of stabilizer.

According to an embodiment, the oral pharmaceutical composition comprises from 1% to 10%, preferably from 2.5% to 7.5% and more preferably from 3.7% to 5% of film coating.

According to another object, the invention relates to a process for the preparation of the oral pharmaceutical composition comprising distinct steps of granulating isoniazid and granulating rifapentine.

According to a specific embodiment, the process for the preparation of a monolayer tablet comprises the steps of:
a) preparing the isoniazid granules,
b) preparing the rifapentine granules,
c) mixing the granules obtained from steps a) and b) with the extragranular excipients,
d) compressing the mixture of step c) to obtain tablets, and
e) film coating the tablets by a method known by the person skilled in the art.

The distinct steps of granulating are performed by wet granulation.

The wet granulation is performed with a granulation composition which can be an aqueous solvent, a liquid binder, an organic solvent, such as isopropyl alcohol, acetone, and chloroform, preferably an aqueous solvent. Said granulation composition can also comprise a binder, a diluent, a disintegrant or mixtures thereof.

After wet granulation, the granules are dried. They can be sifted to improve and enhance the dryness.

The granules can then be sieved to obtain homogenous particle size and to be homogeneously mixed. Preferably, the size of the granules of isoniazid and granules of rifapentine are comprised from 1.3 mm to 0.1 mm, preferably from 1.25 mm to 0.25 mm, more preferably from 1.15 mm to 0.50 mm to be homogeneously mixed.

All the extragranular excipients are mixed together, except the lubricant which is incorporated at the end of the mixing.

Before compression, the mixture can be sieved in order to have homogeneous size particles and thus to facilitate the compression.

When the tablets are formed, they are coated by a method known by the person skilled in the art. The coating is not intended to modify the release of the active substance but to improve its appearance and facilitate its swallowing.

According to a specific embodiment, the process for the preparation of a bilayer tablet comprises the steps of:
a) preparing a layer comprising the isoniazid granules and at least a part of the extragranular excipients,
b) preparing a layer comprising the rifapentine granules and the remaining part of the extragranular excipients,
e) compressing the layer of step a) and the layer of step b) to obtain bilayer tablets, and
f) film coating the tablets by a method known by the person skilled in the art.

The specificities of the different steps described above for the monolayer tablets apply also for the bilayer tablets.

The step of preparing a layer comprises preparing the granules of active principle, then mixing them with the extragranular excipients, optionally followed by a sieving. The present invention will be described with more details in the following examples which are provided for illustrative purposes only.

### EXAMPLES

### Example 1

### Composition of Coated Bilayer Tablets

| | |
|---|---|
| Qty | |
| (mg/tablet) | Function |

Layer with rifapentine granules
Intra-granular excipients

| | | |
|---|---|---|
| rifapentine | 300.00 | active |
| microcrystalline cellulose | 127.50 | diluent |
| sodium starch glycollate | 10.00 | disintegrant |
| pre-gelatinized starch | 40.00 | binder |
| purified water* | q.s | granulation |
| | | fluid |

Extra-granular excipients

| | | |
|---|---|---|
| sodium ascorbate | 5.00 | stabilizer |
| sodium starch glycollate | 10.00 | disintegrant |
| sodium lauryl sulphate | 5.00 | solubilizer |
| calcium stearate | 2.50 | lubricant |

Layer with isoniazid granules
Intra-granular excipients

| | | |
|---|---|---|
| isoniazid | 300.00 | active |
| microcrystalline cellulose | 43.50 | diluent |
| povidone K30 | 10.00 | binder |
| purified water* | q.s | granulation |
| | | fluid |

Extra-granular excipients

| | | |
|---|---|---|
| sodium starch glycollate | 4.00 | disintegrant |
| microcrystalline cellulose | 40.00 | diluent |
| calcium stearate | 2.50 | lubricant |

Film coating

| | | |
|---|---|---|
| PVA pre-mix for coating | 36.000 | film forming |
| | | blend |
| Total (tablet weight) | 936.00 | |

*Removed during drying, does not appear in the final product except in traces.

Process of Preparation of the Coated Bilayer Tablets

The microcrystalline cellulose, pre-gelatinized starch and sodium starch glycollate are separately sifted through, respectively, 0.425 mm, 0.250 mm and 0.180 mm sieve. These materials are then co-sifted with rifapentine through 0.500 mm sieve.

These sifted materials are then dry mixed in a rapid mixer granulator for 20 min at 100 rpm.

They are then granulated in a rapid mixer granulator using purified water initially at 125 rpm and chopper at 1000 rpm for 3 min. The same blend is further kneading at 175 rpm and chopper at 1000 rpm for 6 min and 20 seconds to get the granules of desired consistency.

The obtained wet granules are then dried in a fluid bed dryer at inlet temperature from 55° C. to 60° C. for 4 hours. The resulting dried granules are next sifted through a 0.850 mm sieve to obtain the sifted dried granules.

Sodium ascorbate and sodium starch glycollate are sifted through 0.180 mm sieve and sodium lauryl sulphate is sifted through 0.425 mm sieve. These sifted materials are then blended with the obtained sifted dried granules in a double cone blender for 25 min at 18 rpm speed.

Finally, this blend is lubricated using calcium stearate (sieved through 0.250 mm sieve) for 5 min in double cone blender 18 rpm speed.

The isoniazid and the microcrystalline cellulose are firstly sieved through 0.425 mm sieve and then dry mix in a rapid mixer granulator for 15 min at 75 rpm. This resulting blend is granulated using a solution of povidone K30 dissolved in purified water in a rapid mixer granulator initially at 100 rpm and chopper at 280 rpm for 1.5 min. The same blend is further kneading at 125 rpm and chopper at 500 rpm for 3 min to get the granules of desired consistency

The obtained wet granules are then dried in a fluid bed dryer at inlet temperature from 45° C. to 50° C. for 15 min. The resulting dried granules are then sifted through a 0.600 mm sieve to select the dried granules having a size less than 0.600 mm.

Sodium starch glycollate and microcrystalline cellulose are separately sieved, respectively, through 0.180 mm and 0.425 mm sieve. These sifted materials are then blended in double cone blender with the previously selected dried granules for 15 min at 18 rpm speed.

Finally, this blend is lubricated using calcium stearate (sieved through 0.250 mm sieve) for 5 min in double cone blender 18 rpm speed.

The bilayer tablet is obtained by introducing successively the first blend in the first layer hopper and then the second one in the second layer hopper and compressed as bi-layered tablets using the 20 mm×10.5 mm capsule shaped toolings to obtain the bi-layered tablet of 5.8 mm thickness.

The resulting bilayer tablet is then coated with a aqueous solution of commercially available Opadry II from Colorcon (a readymade pre-mix of PVA polymer with required additive) using an auto coater and with following parameters: Pan speed is from 12 rpm to 14 rpm, spray pump speed is from 2 rpm to 3 rpm, inlet temperature is from 55° C. to 65° C., bed temperature is 36° C. and atomization air pressure is 1 Bar.

Finally the coated bilayer tablet is packed in alu-alu blister.

Stability Data Study of the Packed Coated Bilayer Tablets

The packed coated bilayer tablets were subjected to a stability study at accelerated [40° C./75% RH] and real time condition [25° C./60% RH and 30° C./75% RH]. Analysis by HPLC was carried out just after manufacture (initial), at 3 months and at 6 months. The analysis by HPLC method leads to the total amount of impurities for both rifapentine and isoniazid related substances. Table 1 presents the results of the degradation of rifapentine and isoniazid under these conditions. The results indicated that the total amount of impurities for both rifapentine and isoniazid related substances are below the specification.

TABLE 1
Amount of impurities from rifapentine and isoniazid

| Product Name: Rifapentine & Isoniazid Film Coated Tablets 300/300 mg | | | | | | | | |
|---|---|---|---|---|---|---|---|---|
| Pack Alu-Alu Blister | | | 40° C. + 75% RH | | 25° C./60% RH | | 30° C./75% RH | |
| | Specification | Initial | 3 Month | 6 Month | 3 Month | 6 Month | 3 Month | 6 Month |
| Related Substances - Rifapentine | | | | | | | | |
| MDL 13437(Rifapentine Demetil) | 1.00 | 0.055 | 0.138 | 0.133 | 0.094 | 0.110 | 0.112 | 0.118 |
| MDL 46,863 (Rifapentine N-oxide) | 1.50 | 0.324 | 0.858 | 0.667 | 0.717 | 0.730 | 0.773 | 0.720 |
| MDL 27,718 (25-Desacetyl Rifapentine) | 0.25 | 0.030 | 0.092 | 0.042 | 0.081 | 0.042 | 0.084 | 0.043 |
| MDL 63,746 (3 formyl Rifamycin SV) | 0.80 | 0.070 | 0.160 | 0.230 | 0.117 | 0.144 | 0.124 | 0.157 |
| MDL 105929 (Rifapentine Quinone) | 2.00 | 0.710 | 1.463 | 0.920 | 1.380 | 2.073 | 1.478 | 1.796 |
| Rifapentine + INH Adduct | 4.00 | 0.045 | 0.124 | 0.099 | 0.147 | 0.081 | 0.114 | 0.094 |
| Single Max Unknown | 0.50 | 0.158 | 0.201 | 0.183 | 0.225 | 0.189 | 0.199 | 0.185 |
| Total Impurities | 8.00 | 2.071 | 4.831 | 3.756 | 4.225 | 4.859 | 4.400 | 4.637 |
| Related Substances - Isoniazid | | | | | | | | |
| Single Max Unknown | 0.30 | 0.012 | 0.070 | 0.075 | 0.032 | 0.093 | 0.062 | 0.108 |
| Total Impurities | 2.00 | 0.032 | 0.077 | 0.215 | 0.032 | 0.28 | 0.071 | 0.436 |

### Example 2

### Composition of Coated Monolayer Tablets

| | |
|---|---|
| Qty | |
| (mg/tablet) | Function |

Intra-granular excipients
Rifapentine granules

| | | |
|---|---|---|
| rifapentine | 300.00 | active |
| microcrystalline cellulose | 97.50 | diluent |
| sodium starch glycollate | 10.00 | disintegrant |
| pre-gelatinized starch | 35.00 | binder |
| purified water* | q.s | granulation |
| | | fluid |

Isoniazid granules

| | | |
|---|---|---|
| isoniazid | 300.00 | active |
| microcrystalline cellulose | 20.00 | diluent |
| povidone K30 | 3.00 | binder |
| purified water* | q.s | granulating |
| | | fluid |

Extra-granular excipients

| | | |
|---|---|---|
| sodium ascorbate | 5.00 | stablilizer |
| sodium starch glycollate | 22.00 | disintegrant |
| sodium lauryl sulphate | 5.00 | solubilizer |
| calcium stearate | 2.50 | lubricant |

Film coating

| | | |
|---|---|---|
| HPMC pre-mix | 37.25 | film forming |
| | | blend |
| di-sodium EDTA | 0.25 | stabilizer |
| sodium ascorbate | 2.50 | stabilizer |
| Total (tablet weight) | 840 | |

*Removed during drying, does not appear in the final product except in traces.

Process of Preparation of the Coated Monolayer Tablets

The granules are prepared as disclosed in example 1 but using the constituents mentioned in the above table.

The rifapentine and isoniazid selected dried granules are firstly blended with the extra-granular excipients: sodium ascorbate, sodium starch glycollate and sodium lauryl sulphate. The resulting blend is then lubricated using calcium stearate. Finally, the lubricated blend is compressed into round tablets using 14 mm round standard concave tooling in monolayer compression machine. The diameter and the thickness of the resulting monolayer tablet are 14 mm and 6.30 mm respectively

The monolayer tablet is then coated with an aqueous solution of dissolved di-sodium EDTA, sodium ascorbate and of commercially available Opadry (Colorcon, India Ltd, a readymade pre-mix of HPMC polymer with required additives) using auto-coater with following parameters: Pan speed is from 4 rpm to 6 rpm, spray pump speed is from 1 rpm to 6 rpm, inlet temperature is around 70, bed temperature is around 38° C. and atomization air pressure is around 1 bar.

Finally the coated monolayer tablet is packed in alu-alu Blister.

The packed coated monolayer tablets were subjected to a stability study as in example 1. Table 2 presents the degradation of rifapentine and isoniazid under these conditions. The results indicate that the total amount of impurities for both rifapentine and isoniazid related substances are below the specification.

TABLE 2
Amount of impurities from rifapentine and isoniazid

| Product Name: Rifapentine & Isoniazid Film Coated Tablets 300/300 mg | | | | | | | | |
|---|---|---|---|---|---|---|---|---|
| Pack Alu-Alu Blister | | | | | | | | |
| | Specification | | 40° C./75% RH | | 25° C./60% RH | | 30° C./75% RH | |
| | % | Initial | 3 Month | 6 Month | 3 Month | 6 Month | 3 Month | 6 Month |
| Related Substances - Rifapentine | | | | | | | | |
| MDL 13437(Rifapentine Demetil) | 1.00 | 0.045 | 0.178 | 0.132 | 0.141 | 0.104 | 0.156 | 0.125 |
| MDL 46,863 (Rifapentine N-oxide) | 1.50 | 0.226 | 1.193 | 0.748 | 1.078 | 0.824 | 1.134 | 0.723 |
| MDL 27,718 (25-Desacetyl Rifapentine) | 0.25 | 0.014 | 0.022 | 0.016 | 0.022 | 0.045 | 0.025 | 0.016 |
| MDL 63,746 (3 formyl Rifamycin SV) | 0.80 | 0.018 | 0.031 | 0.008 | 0.026 | 0.021 | 0.029 | 0.020 |
| MDL 105929 (Rifapentine Quinone) | 3.00 | 0.019 | 0.260 | 0.025 | 0.250 | 0.176 | 0.232 | 0.049 |
| RPT + INH Adduct | 4.00 | 0.055 | 0.564 | 0.686 | 0.165 | 0.205 | 0.286 | 0.379 |
| Single Max Unknown | 0.50 | 0.245 | 0.564 | 0.192 | 0.157 | 0.195 | 0.286 | 0.485 |
| Total Impurities | 8.00 | 1.266 | 3.705 | 2.946 | 3.021 | 2.607 | 3.127 | 2.438 |
| Related Substances - Isoniazid | | | | | | | | |
| Single Max Unknown | 0.30 | 0.039 | 0.060 | 0.119 | 0.047 | 0.114 | 0.053 | 0.109 |
| Total Impurities | 2.00 | 0.039 | 0.163 | 0.298 | 0.119 | 0.307 | 0.131 | 0.286 |

1. An oral pharmaceutical fixed dose composition for use in the treatment of tuberculosis, said oral pharmaceutical composition comprising:
a) granules comprising isoniazid and at least one intragranular excipient,
b) granules comprising rifapentine and at least one intragranular excipient, and
c) at least one extragranular excipient.

2. An oral pharmaceutical composition according to claim 1, wherein said oral pharmaceutical composition is chemically stable.

3. An oral pharmaceutical composition according to claim 1 or 2, wherein said oral pharmaceutical composition is in the form of a coated tablet.

4. An oral pharmaceutical composition according to any one of the claims 1 to 3, wherein said oral pharmaceutical composition is in the form of a coated bilayer tablet comprising:
a layer comprising isoniazid granules (a) and at least one extragranular excipient,
a layer comprising rifapentine granules (b) and at least one extragranular excipient, and
a film coating.

5. An oral pharmaceutical composition according to any one of the claims 1 to 4, wherein the ratio of rifapentine to isoniazid is comprised from 5:1 to 1:0.5, preferably the ratio is 1:1.

6. A process for the preparation of an oral pharmaceutical composition according to any one of the claims 1 to 5, characterized in that it comprises distinct steps of granulating isoniazid and granulating rifapentine.

7. A process according to claim 6, characterized in that the preparation of the granules is made by wet granulation, preferably in an aqueous solvent.

8. A process according to claim 6 or 7, characterized in that it comprises the steps of:
a) preparing the isoniazid granules,
b) preparing the rifapentine granules,
c) mixing the granules obtained from steps a) and b) with the extragranular excipients,
d) compressing the mixture of step c) to obtain tablets, and
e) film coating the tablets.

9. A process according to claims 6 to 8, characterized in that it comprises the steps of:
a) preparing the isoniazid granules,
b) mixing the granules obtained from step a) with at least a part of the extragranular excipients,
c) preparing the rifapentine granules,
d) mixing the granules obtained from step c) with the remaining part of the extragranular excipients,
e) compressing the mixture of steps b) and d) to obtain bi-layer tablets, and
f) film coating the tablets.
